(19) United States
(12) Patent Application Publication
(10) Pub. No.: US 2019/0117722 A1
(43) Pub. Date: Apr. 25, 2019

(54) COMPOSITION FOR TREATING ALOPECIA AND METHODS OF MAKING AND USING THE SAME

(71) Applicants: Theresa Hackney Spiller, Pearland, TX (US); Raphael Nicholas Spiller, Pearland, TX (US)

(72) Inventors: Theresa Hackney Spiller, Pearland, TX (US); Raphael Nicholas Spiller, Pearland, TX (US)

(21) Appl. No.: 16/165,323

(22) Filed: Oct. 19, 2018

Related U.S. Application Data
(60) Provisional application No. 62/574,246, filed on Oct. 19, 2017.

(51) Int. Cl. A61K 36/886; A61K 9/00; A61K 31/047; A61K 36/47; A61K 31/10; A61K 31/4188; A61K 47/46; A61P 17/14; A61K 36/63; A61K 36/87; A61K 36/54; A61K 36/53; A61K 36/752; A61K 36/328; A61K 36/324; A61K 36/534; A61K 36/736; A61K 36/286; A61K 36/30; A61K 36/185

(52) U.S. Cl. CPC A61K 36/886; A61K 36/185; A61K 31/047; A61K 36/47; A61K 31/10; A61K 31/4188; A61K 47/46; A61P 17/14; A61K 36/63; A61K 36/87; A61K 36/54; A61K 36/53; A61K 36/752; A61K 36/328; A61K 36/324; A61K 36/534; A61K 36/736; A61K 36/286; A61K 36/30; A61K 9/0014

(57) ABSTRACT

The present disclosure relates to topical compositions for treating alopecia and methods of making and administering such compositions. In some embodiments, the topical composition can include a mixture of an aloe vera extract, glycerin, a castor oil, methylsulfonylmethane, biotin, or mixtures thereof. Benefits of the compositions and methods of treatment can include normal regrowth of hair.

### RELATED APPLICATIONS

This application claims priority to U.S. Provisional Patent Application 62/574,246 filed on Oct. 19, 2017 which is specifically incorporated by reference in its entirety herein.

### FIELD

The present disclosure relates to compositions for treating alopecia and methods of making and administering such compositions. Benefits of the compositions and methods of treatment can include regrowth of normal hair or partial restoration of normal hair.

### BACKGROUND

Alopecia is a type of hair loss that is believed to occur when the body's immune system attacks the hair follicles, resulting in portions of hair falling out. For those suffering from alopecia, the hair loss can range from bald patches on the scalp to total baldness. In some cases, the hair from a bald patch will regrow over time, but the hair can be thinner, less dense, and/or whitish in appearance. In many cases, the hair from one bald patch will regrow in one spot, only to fall out in another. In other cases, the hair loss can be permanent. There are different types of alopecia, which include alopecia totalis (total hair loss on head), alopecia universalis (all body hair is loss), androgenetic alopecia (male-patterned baldness), alopecia areata (hair patches falling out), diffuse alopecia (sudden radical hair loss), postpartum alopecia (temporary hair loss after pregnancy or delivery), alopecia barbae (hair loss in the beard area), and more. Also, alopecia is known to occur in humans and other mammals, such as dogs, cats, horses, and the like.

The cosmetic industry has devoted millions of dollars and countless hours of research to solve this problem. Yet the cause of alopecia is not well understood. There is no known cure for alopecia. The diagnosis of alopecia typically occurs through physical examination of the bald spots, microscopic examination of hair samples, and/or blood tests to eliminate other causes, such as an over or under active thyroid gland. One of the most common treatments for alopecia includes painful and expensive injections of corticosteroids into the scalp or skin, about 1 cm apart, every 4 to 6 weeks. Other common treatments for alopecia include the application of topical corticosteroids, such as minoxidil (ROGAINE®), which is often ineffective, expensive, and includes unnatural ingredients. Another type of treatment includes irritation methods, such as the application of anthralin (dithranol) directly to the scalp. Irritation methods are known to impart a burning, irritating sensation. They are also known to have the look and feel of having the scalp painted with tar.

There is a need for topical compositions and treatments that are effective in treating alopecia. There is a need for topical compositions and treatments that include only safe, natural ingredients. There is a need for topical compositions and treatments that look and/or feel like a normal hair gel or a normal cosmetic product.

### SUMMARY

The present disclosure relates to compositions for treating alopecia and methods of making and administering such compositions. In some embodiments, a topical composition for treating alopecia includes a mixture of a first substance and a second substance, wherein the first substance includes an aloe vera extract, and wherein the second substance includes glycerin, a castor oil, methylsulfonylmethane, biotin, or mixtures thereof. In some embodiments of the topical composition, the second substance includes glycerin, a castor oil, or mixtures thereof. In some embodiments of the topical composition, the second substance includes glycerin. In some embodiments of the topical composition, the second substance includes the castor oil. In some embodiments of the topical composition, the first substance is contained in an amount of from about 0.1 volume percent to about 80 volume percent based on a total volume of the mixture. In some embodiments of the topical composition, the second substance is contained in an amount of from about 0.1 volume percent to about 80 volume percent based on a total volume of the mixture. In some embodiments of the topical composition, the first substance and the second substance are contained in a volume ratio of from about 2:1 to about 1:4. In some embodiments, the topical composition further includes a third substance selected from the group consisting of an olive oil, an alkaline water, a grape seed oil, an avocado oil, a rosemary oil, a grape fruit oil, a myrrh, a frankincense oil, a lavender oil, a peppermint oil, a sweet almond oil, a safflower seed oil, a borage seed oil, a Haitian black castor oil, an argan oil, and mixtures thereof. In some embodiments, the third substance is contained in an amount of from about 0.1 volume percent to about 70 volume percent based on a total volume of the mixture. In some embodiments, the topical composition further includes a base substance. In some embodiments, the base substance includes a tallow. In an embodiment, the base substance includes shae butter.

In some embodiments, the topical composition for treating alopecia includes, a mixture of a first substance and a second substance, wherein the first substance includes a castor oil, and wherein the second substance includes methylsulfonylmethane, glycerin, an aloe vera extract, biotin, or mixtures thereof. In some embodiments, the topical composition for treating alopecia includes a mixture of a first substance and a second substance, wherein the first substance includes glycerin, and wherein the second substance includes a castor oil, an aloe vera extract, methylsulfonylmethane, biotin, or mixtures thereof.

A method for treating a subject suffering from and/or diagnosed with alopecia is disclosed. In some embodiments, the method for treating a subject suffering from and/or diagnosed with alopecia includes applying a dosage of a topical composition to an affected area of the subject, wherein the topical composition includes a mixture of first substance and a second substance; the first substance includes an aloe vera extract; and the second substance includes glycerin, a castor oil, methylsulfonylmethane, biotin, or mixtures thereof. In some embodiments of the method, the dosage contains about 0.3 ml to about 20 ml of the topical composition. In some embodiments of the method, the dosage is applied from one to four times daily for a period of from 1 to 12 weeks. In some embodiments of the method, the dosage is applied for a first course of treatment, treatment is discontinued for a period of time, and the treatment is resumed for at least a second course of treatment. In some embodiments of the method, the treatment is discontinued for a period of days, weeks, one month, two months, three months, or longer. In some embodiments of the method, the dosage is applied to the affected area in an amount of, at a frequency, and for a total number of applications sufficient to provide therapeutic benefit. In some embodiments of the method, the second substance includes glycerin, a castor oil, or a mixture thereof.

The foregoing has outlined rather broadly the features of the present disclosure in order that the detailed description that follows may be better understood. Additional features and advantages of the disclosure will be described hereinafter, which form the subject of the claims.

### BRIEF DESCRIPTION OF THE DRAWINGS

In order that the manner in which the above-recited and other enhancements and objects of the disclosure are obtained, a more particular description of the disclosure briefly described above will be rendered by reference to specific embodiments thereof which are illustrated in the appended drawings. Understanding that these drawings depict only typical embodiments of the disclosure and are therefore not to be considered limiting of its scope, the disclosure will be described with additional specificity and detail through the use of the accompanying drawings in which:

FIG. 1 is a picture of a woman suffering from alopecia areata before using a composition or treatment disclosed herein.

FIG. 2 is a picture of the same woman as FIG. 1 after undergoing treatment using an embodiment of the topical compositions and treatments methods disclosed herein 1-2 per day for 4 months.

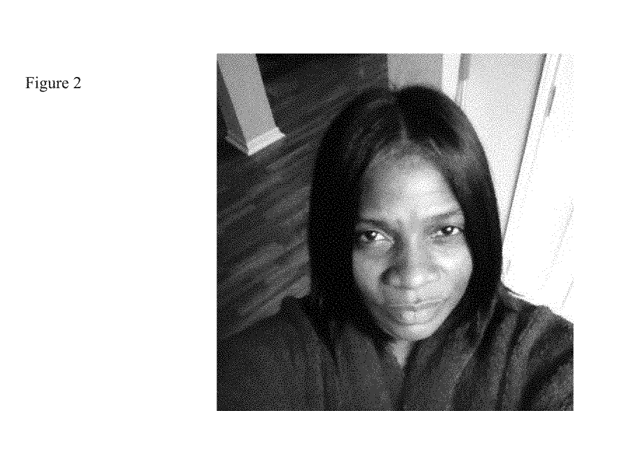

FIG. 3 is a picture of a young girl suffering from a bacterial infection before using a composition or treatment disclosed herein.

FIG. 4 is a picture of the same young girl as FIG. 3 after undergoing treatment using an embodiment of the topical compositions and treatments methods disclosed herein 1-2 per day for 3 months.

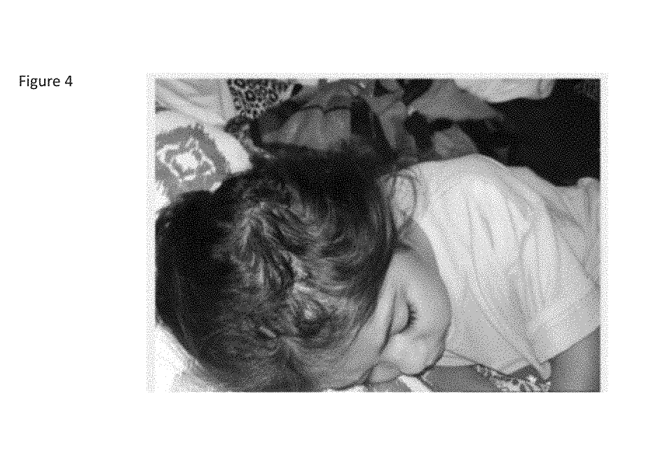

FIG. 5 is a picture of a woman suffering from hair thinning before using a composition or treatment disclosed herein.

FIG. 6 is a picture of the same woman as FIG. 5 after undergoing treatment using an embodiment of the topical compositions and treatments methods disclosed herein 1-2 per day for 4 months.

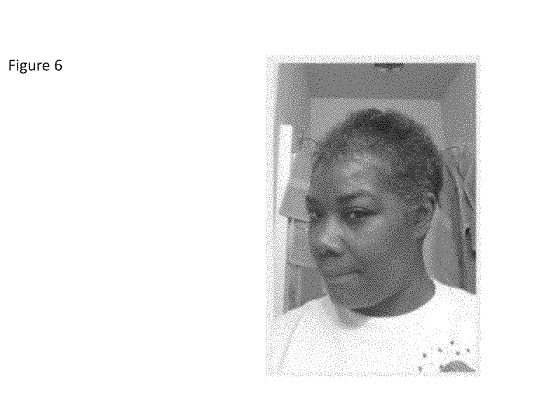

### DETAILED DESCRIPTION

The particulars shown herein are by way of example and for purposes of illustrative discussion of the preferred embodiments of the present disclosure only and are presented in the cause of providing what is believed to be the most useful and readily understood description of the principles and conceptual aspects of various embodiments of the disclosure. In this regard, no attempt is made to show structural details of the disclosure in more detail than is necessary for the fundamental understanding of the disclosure, the description taken with the drawings making apparent to those skilled in the art how the several forms of the disclosure may be embodied in practice.

The following definitions and explanations are meant and intended to be controlling in any future construction unless clearly and unambiguously modified in the following examples or when application of the meaning renders any construction meaningless or essentially meaningless. In cases where the construction of the term would render it meaningless or essentially meaningless, the definition should be taken from Webster's Dictionary 3^{rd} Edition.

Unless otherwise noted, all measurements are in standard metric units.

Unless otherwise noted, all instances of the words “a,” “an,” or “the” can refer to one or more than one of the word that they modify.

Unless otherwise noted, the phrase “at least one of” means one or more than one of an object. For example, “at least one of H₁ and H₂” means H₁, H₂, or both.

Unless otherwise noted, the term “about” refers to ±10% of the non-percentage number that is described, rounded to the nearest whole integer. For example, about 100 mm, would include up to 90 to 110 mm. Unless otherwise noted, the term “about” refers to ±3% of a percentage number. For example, about 20% would include up to 17 to 23%. When the term “about” is discussed in terms of a range, then the term refers to the appropriate amount less than the lower limit and more than the upper limit. For example, from about 100 ml to about 200 ml would include up to from 90 ml to 220 ml.

Unless otherwise noted, the term “topical” means related to or applied directly to the skin or scalp.

Unless otherwise noted, the term “animal” means a non-human mammal.

In some embodiments, the present disclosure relates to a topical composition for treating alopecia. Just as the cause of alopecia is not well understood, the medical mechanism why or how the topical compositions are effective for treating alopecia is also not well understood. These compositions were surprisingly discovered when a non-scientist was diagnosed with alopecia areata, and set out to find a combination of natural products that could be safely and comfortably applied directly to the scalp or skin to cure or reduce the symptoms of alopecia areata. Through blind trial and error, the topical compositions for treating alopecia were discovered by mixing various substances known to be safe for topical application and known to have at least one beneficial and/or medicinal property. Research is currently under way to develop a better understanding of the topical compositions and the mechanism of their efficacy.

In some embodiments, the present disclosure relates to a topical compositions for treating alopecia. In some embodiments, the topical composition includes a mixture of a first substance and a second substance, wherein the first substance includes an aloe vera extract, and wherein the second substance includes glycerin, a castor oil, methylsulfonylmethane, biotin, or mixtures thereof. In some embodiments, the topical composition includes a mixture of a first substance and a second substance, wherein the first substance includes a castor oil, and wherein the second substance includes methylsulfonylmethane, glycerin, aloe vera extract, biotin, or mixtures thereof. In some embodiments, the topical composition includes a mixture of a first substance and a second substance, wherein the first substance includes glycerin, and wherein the second substance includes a castor oil, an aloe vera extract, methylsulfonylmethane, biotin, or mixtures thereof.

In some embodiments, the first substance is contained in an amount of from 0.1 volume percent to about 95 volume percent based on a total volume of the mixture. In some embodiments, the first substance is contained in an amount of from about 5 volume percent to about 80 volume percent, including from about 5 volume percent to about 50 volume percent, including from about 5 to 30 volume percent, based on a total volume of the mixture. In some embodiments, the first substance is one of an aloe vera extract, glycerin, a castor oil, methylsulfonylmethane, or biotin.

In some embodiments, the second substance is contained in an amount of from 0.1 volume percent to about 95 volume percent based on a total volume of the mixture. In some embodiments, the second substance is contained in an amount of from about 5 volume percent to about 80 volume percent, including from about 5 volume percent to about 50 volume percent, including from about 5 to about 30 volume percent, based on a total volume of the mixture. In some embodiments, the second substance is one or more of an aloe vera extract, glycerin, a castor oil, methylsulfonylmethane, or biotin, provided that the second substance is not the same as the first substance.

In some embodiments, the first substance and the second substance are contained in a volume ratio of from about 2:1 to about 1:4, including from about 1:1 to about 1:2, including from about 1:1 to about 1:1.5.

In some embodiments, the aloe vera extract can include any compound, substance, or mixture thereof, which is capable of being removed from the aloe vera plant, even if the compound, substance, or mixture thereof is not obtained from a non-aloe vera source. In some embodiments, the aloe vera extract includes aloe vera juice or aloe vera gel. In some embodiments, the aloe vera extract includes an aloe vera gel having from about 90% to 100% purity. In some embodiments, aloe vera extracts can include an amino acid, an anthraquinone, an enzyme, a hormone, a lignin, salicylic acid, a saponin, a sterol, a vitamin, a mineral, a sugar, or combinations thereof.

Suitable aloe vera extracts can include aloe vera juice or aloe vera gel. Suitable aloe vera extracts can include a commercially available aloe vera gel.

Suitable aloe vera extracts can include amino acids, including isoleucine, leucine, lysine, methionine, phenylalanine, threonine, valine, alanine, arginine, asparagine, cysteine, glutamic acid, glycine, histidine, proline, serine, tyrosine, glutamine, and/or aspartic acid. Suitable aloe vera extracts can include anthraquinones, such as aloe emodin, aloetic acid, aloin, antracin, antranol, barbaloin, chrysophanic acid, emodin, ethereal oil, cinnamonic acid ester, isobarbaloin, and/or resistannol. Suitable aloe vera extracts can include enzymes, such as aliiase, alkaline phosphatase, amylase, carboxypeptidase, catalase, cellulase, lipase, and/or peroxidase. Suitable aloe vera extracts can include hormones, such as auxin and/or gibberellin. Suitable aloe vera extracts can include sterols, including cholesterol, campesterol, lupeol, and/or sitosterol. Suitable aloe vera extracts can include vitamins, such as vitamin A (beta-carotene), vitamin B1 (thiamine), vitamin B2 (riboflavin), vitamin B3 (niacin), vitamin B5, vitamin B6 (pyridoksin), vitamin B12, vitamin C, vitamin E, kolin, folate, or combinations thereof. Suitable aloe vera extracts can include sugars and/or polysaccharides, such sugars include glucose and fructose; and such polysaccharides include acemannan. Suitable aloe vera extracts can include a lignin, salicylic acid, a saponin, and/or minerals. Suitable minerals include calcium, chrome, copper, iron, magnesium, manganese, potassium, phosphorus, sodium, or zinc, or combinations thereof. Suitable aloe vera extracts can include phytochemicals, such as acetylated mannans, polymannans, anthraquinone c-glycosides, anthrones, or mixtures thereof; and anthraquinones, such as emodin and various lectins.

In some embodiments, the topical composition includes glycerin, which is also known as “glycerine” or glycerol (C₃H₈O₃).

In some embodiments, the topical composition includes a castor oil, which includes any extract from the seeds of the castor oil plant (Ricinus communis). Suitable extracts from the seeds of the castor oil plant can include ricinoleic acid, oleic acid, linoleic acid, α-linolenic acid, stearic acid, palmitic acid, and dihydroxystearic acid.

In some embodiments, the topical composition includes methylsulfonylmethane (MSM, C₂H₆O₂S), which is also known as dimethyl sulfone.

In some embodiments, the topical composition includes biotin (5-[(3aS,4S,6aR)-2-oxohexahydro-1H-thieno [3,4-d]imidazol-4-yl]pentanoic acid; C₁₀H₁₆N₂O₃S). Biotin is also known as vitamin B7, vitamin H, or coenzyme R.

In some embodiments, the topical composition can include a third substance selected from the group consisting of an olive oil, an alkaline water, a grape seed oil, an avocado oil, a rosemary oil, a grape fruit oil, a myrrh, a frankincense oil, a lavender oil, a peppermint oil, a almond oil, a sweet almond oil, a safflower seed oil, a borage seed oil, a Haitian black castor oil, an argan oil, and/or mixtures thereof. In some embodiments, the topical composition can include the third substance in an amount of from 0.1 volume percent to about 70 volume percent based on a total volume of the mixture. In some embodiments, the topical composition can include the third substance in an amount of from 0.3 volume percent to about 50 volume percent, including from 1 volume percent to about 30 volume percent, including from 0.3 to about 7 volume percent, based on a total volume of the mixture.

In some embodiments, the topical composition includes a base substance, such as tallow. In some embodiments, the topical composition includes a base substance, such as shae butter.

In some embodiments, the topical composition includes a mixture containing the following substances:

| Substance | Vol (ml) | Vol % of Total |
|---|---|---|
| aloe vera gel | 118 | 8.5 |
| glycerin oil | 118 | 8.5 |
| olive oil | 118 | 8.5 |
| alkaline water | 118 | 8.5 |
| grape seed oil | 118 | 8.5 |
| avocado oil | 118 | 8.5 |
| methylsulfonylmethane | 5 | 0.4 |
| rosemary oil | 59 | 4.2 |
| grape fruit oil | 59 | 4.2 |
| Biotin (10 capsule in 5 ml) | 5 | 0.4 |
| myrrh oil | 5 | 0.4 |
| frankincense oil | 5 | 0.4 |
| lavendar oil | 5 | 0.4 |
| peppermint oil | 59 | 8.5 |
| sweet almond oil | 118 | 8.5 |
| safflower oil | 118 | 8.5 |
| castor oil | 118 | 8.5 |
| borage oil | 118 | 8.5 |
| Haitian black castor oil | 5 | 0.4 |
| argan oil | 5 | 0.4 |
| Total of Substances | 1392 | 100.0 |

In some embodiments, the topical composition takes the form of or is contained in a cream, a gel, a foam, an ointment, a paste, a lotion, a liquid, a shampoo, a conditioner, a hairspray, a hair muse, a hair dye, or other topical product formulated for or approved for human use. In some embodiments, the topical composition takes the form of or is contained in an animal shampoo, an animal conditioner, or an animal pest control product, such as a flea and tick spray, or any other topical product formulated for or approved for animal care.

In some embodiments, the topical composition is formulated or approved for use on a mammal, wherein the mammal is a cat, a dog, or a horse.

In some embodiments, a method is disclosed for treating, preventing, and/or curing alopecia, including alopecia totalis (total hairloss on head), alopecia universalis (all body hair is loss), androgenetic alopecia (male-patterned baldness), alopecia areata (hair patches falling out), diffuse alopecia (sudden radical hairloss), postpartum alopecia (temporary hairloss after pregnancy or delivery), alopecia barbae (hairloss in the beard area), patterned baldness, and/or hair loss in a human or animal subject. In some embodiments, a method is disclosed for alleviating or reducing the symptoms of alopecia, patterned baldness, and/or hair loss in a human or animal subject. In some embodiments, the method includes applying a dosage of a topical composition, wherein the topical composition includes a mixture of a first substance and a second substance; the first substance includes an aloe vera extract; and the second substance includes glycerin, a castor oil, methylsulfonyl-methane, biotin, or mixtures thereof. In some embodiments, the method includes applying a dosage of a topical composition, wherein the topical composition includes a mixture of first substance and a second substance; the first substance includes a glycerin; and the second substance includes an aloe vera extract, a castor oil, methylsulfonylmethane, biotin, or mixtures thereof. In some embodiments, the method includes applying a dosage of a topical composition, wherein the topical composition includes a mixture of first substance and a second substance; the first substance includes methylsulfonylmethane; and the second substance includes an aloe vera extract, a castor oil, biotin, or mixtures thereof.

In certain embodiments, the topical composition can be applied directly to the dermis and/or hair, including directly to the scalp, skin, or hair.

In certain embodiments, the method of treating alopecia in a subject in need thereof is disclosed, wherein the subject is a human or a mammal. In certain embodiments of the method, the mammal can include a cat, a dog, or a horse, or any other mammal in which alopecia is known to occur. In certain embodiments, the diagnosis can be provided by a doctor, nurse, veterinarian, or other healthcare worker.

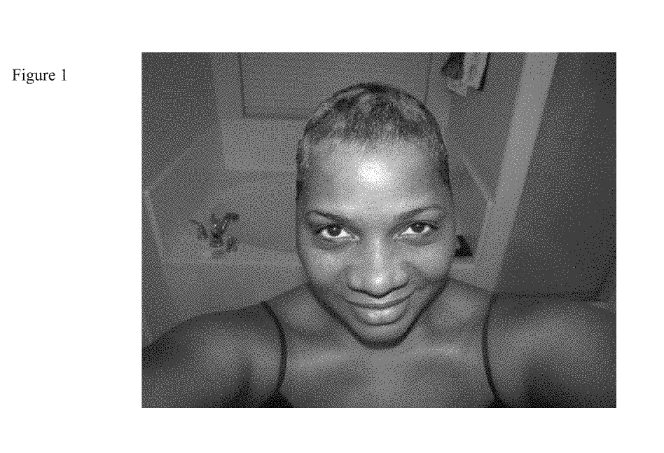

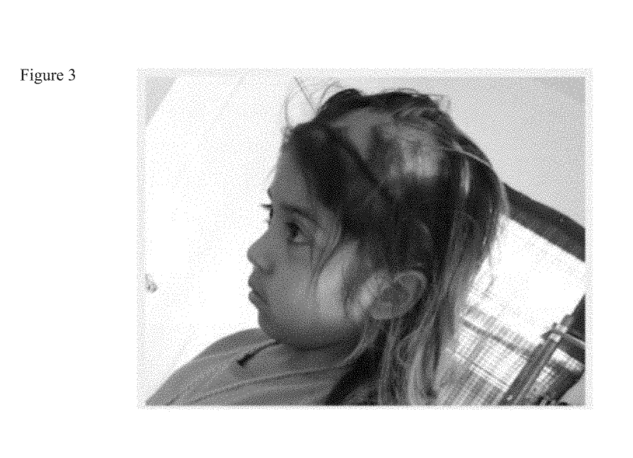

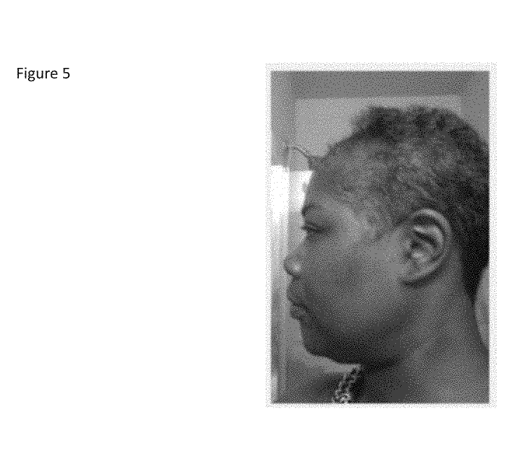

In certain embodiments, the topical composition contains a dosage for about 0.3 ml to about 20 ml of the topical composition, including from 2 ml to about 10 ml, including from 2 ml to 5 ml. In certain embodiments, the topical composition is applied from 1, 2, 3, or 4 times daily for a period of from 1 to 12 weeks, including from 2 to 8 weeks, including from 2 to 4 weeks. In certain embodiments, the dosage is applied for a first course of treatment, treatment is discontinued for a period of time, and the treatment is resumed for at least a second course of treatment. In certain embodiments, the treatment is discontinued for a period of days, weeks, one month, two months, three months, or longer. In certain embodiments, the dosage is applied to the affected area in an amount of, at a frequency, and for a total number of applications sufficient to provide therapeutic benefit. For example, beneficial results have been observed in a human subject from a single course of daily treatment for a duration of four weeks. Comparing Figures FIGS. 1, 3, and 5 (before pictures) to 2, 4, and 6 (after pictures), respectively, some subjects have experienced complete or partial restoration of normal hair after a series of treatments by applying dosages of an embodiment of the topical compositions disclosed herein.

### EXAMPLES

### Example 1

An embodiment of the topical compositions disclosed herein can be prepared as follows: mix the following commercially available substances in a blender and add tallow until a desirable consistency is reached. In an embodiment, 4 cups of tallow are added.

| Substance | Vol (ml) | Vol % of Total |
|---|---|---|
| aloe vera gel | 118 | 8.5 |
| glycerin oil | 118 | 8.5 |
| olive oil | 118 | 8.5 |
| alkaline water | 118 | 8.5 |
| grape seed oil | 118 | 8.5 |
| avocado oil | 118 | 8.5 |
| methylsulfonylmethane | 5 | 0.4 |
| rosemary oil | 59 | 4.2 |
| grape fruit oil | 59 | 4.2 |
| Biotin (10 capsule in 5 ml) | 5 | 0.4 |
| myrrh oil | 5 | 0.4 |
| frankincense oil | 5 | 0.4 |
| lavendar oil | 5 | 0.4 |
| peppermint oil | 59 | 8.5 |
| sweet almond oil | 118 | 8.5 |
| safflower oil | 118 | 8.5 |
| castor oil | 118 | 8.5 |
| borage oil | 118 | 8.5 |
| Haitian black castor oil | 5 | 0.4 |
| argan oil | 5 | 0.4 |
| Total of Substances | 1392 | 100.0 |

A few milliliters of the topical compositions was applied to the scalp 1-2 times per day after the hair was cleaned with shampoo and rinsed. This dosage was repeated for 3-4 months before the results shown in the Figures.

### Example 2

An embodiment of the topical compositions disclosed herein can be prepared as follows: mix the following commercially available substances in a blender and add shae butter until a desirable consistency is reached. In an embodiment, 4 cups of shae butter are added.

| Substance | Vol (ml) | Vol % of Total |
|---|---|---|
| aloe vera gel | 118 | 8.5 |
| glycerin oil | 118 | 8.5 |
| olive oil | 118 | 8.5 |
| alkaline water | 118 | 8.5 |
| grape seed oil | 118 | 8.5 |
| avocado oil | 118 | 8.5 |
| methylsulfonylmethane | 5 | 0.4 |
| rosemary oil | 59 | 4.2 |
| grape fruit oil | 59 | 4.2 |
| Biotin (10 capsule in 5 ml) | 5 | 0.4 |
| myrrh oil | 5 | 0.4 |
| frankincense oil | 5 | 0.4 |
| lavendar oil | 5 | 0.4 |
| peppermint oil | 59 | 8.5 |
| sweet almond oil | 118 | 8.5 |
| safflower oil | 118 | 8.5 |
| castor oil | 118 | 8.5 |
| borage oil | 118 | 8.5 |
| Haitian black castor oil | 5 | 0.4 |
| argan oil | 5 | 0.4 |
| Total of Substances | 1392 | 100.0 |

A few milliliters of the topical compositions was applied to the scalp 1-2 times per day after the hair was cleaned with shampoo and rinsed. This dosage was repeated for 3-4 months before the results shown in the Figures.

All of the compositions and methods disclosed and claimed herein can be made and executed without undue experimentation in light of the present disclosure. While the compositions and methods of this disclosure have been described in terms of preferred embodiments, it will be apparent to those of skill in the art that variations may be applied to the compositions and methods and in the steps or in the sequence of steps of the methods described herein without departing from the concept, spirit and scope of the disclosure. More specifically, it will be apparent that certain agents which are both chemically related may be substituted for the agents described herein while the same or similar results would be achieved. All such similar substitutes and modifications apparent to those skilled in the art are deemed to be within the spirit, scope and concept of the disclosure as defined by the appended claims.

What is claimed is:

1. A topical composition for treating alopecia comprising:
a mixture of a first substance and a second substance,
wherein the first substance includes an aloe vera extract, and
wherein the second substance includes glycerin, a castor oil, methylsulfonylmethane, biotin, or mixtures thereof.

2. The topical composition of claim 1, wherein the second substance includes glycerin, a castor oil, or mixtures thereof.

3. The topical composition of claim 1, wherein the second substance includes glycerin.

4. The topical composition of claim 1, wherein the second substance includes the castor oil.

5. The topical composition of claim 1, wherein the first substance is contained in an amount of from about 0.1 volume percent to about 80 volume percent based on a total volume of the mixture.

6. The topical composition of claim 1, wherein the second substance is contained in an amount of from about 0.1 volume percent to about 80 volume percent based on a total volume of the mixture.

7. The topical composition of claim 1, wherein the first substance and the second substance are contained in a volume ratio of from about 2:1 to about 1:4.

8. The topical composition of claim 1, further comprising a third substance selected from the group consisting of an olive oil, an alkaline water, a grape seed oil, an avocado oil, a rosemary oil, a grape fruit oil, a myrrh, a frankincense oil, a lavender oil, a peppermint oil, a sweet almond oil, a safflower seed oil, a borage seed oil, a Haitian black castor oil, an argan oil, and mixtures thereof.

9. The topical composition of claim 8, wherein the third substance is contained in an amount of from about 0.1 volume percent to about 70 volume percent based on a total volume of the mixture.

10. The topical composition of claim 1, further comprising a base substance.

11. The topical composition of claim 1, wherein the base substance includes shae butter.

12. A topical composition for treating alopecia comprising:
a mixture of a first substance and a second substance,
wherein the first substance includes a castor oil, and
wherein the second substance includes methylsulfonylmethane, glycerin, aloe vera extract, biotin, or mixtures thereof.

13. A topical composition for treating alopecia comprising:
a mixture of a first substance and a second substance,
wherein the first substance includes glycerin, and
wherein the second substance includes a castor oil, an aloe vera extract, methylsulfonylmethane, biotin, or mixtures thereof.

14. A method for treating a subject suffering from and/or diagnosed with alopecia comprising:
applying a dosage of the topical composition of claim 1 to an affected area of the subject.

15. The method of claim 14, wherein the dosage contains about 0.3 ml to about 20 ml of the topical composition.

16. The method of claim 14, wherein the dosage is applied from one to four times daily for a period of from 1 to 12 weeks.

17. The method of claim 14, wherein the dosage is applied for a first course of treatment, treatment is discontinued for a period of time, and the treatment is resumed for at least a second course of treatment.

18. The method of claim 17, wherein the treatment is discontinued for a period of days, weeks, one month, two months, three months, or longer.

19. The method of claim 14, where the dosage is applied to the affected area in an amount of, at a frequency, and for a total number of applications sufficient to provide therapeutic benefit.

20. The method of claim 14, wherein the second substance includes glycerin, a castor oil, or a mixture thereof.
